(19) United States
(12) Patent Application Publication
(10) Pub. No.: US 2021/0259332 A1
(43) Pub. Date: Aug. 26, 2021

(54) CLOTHING ARTICLE COMPRISING A TOURNIQUET

(71) Applicant: Jerry Allen Nance, II, Gadsden, TN (US)

(72) Inventor: Jerry Allen Nance, II, Gadsden, TN (US)

(21) Appl. No.: 17/181,602

(22) Filed: Feb. 22, 2021

Related U.S. Application Data
(60) Provisional application No. 62/979,374, filed on Feb. 20, 2020.

(51) Int. Cl. A41D 13/00; A61B 17/132; A41D 1/04; A41B 1/08; A62B 35/00

(52) U.S. Cl. CPC A41D 13/0007; A61B 17/1322; A41D 1/04; A41B 2400/32; A62B 35/0006; A41D 2400/32; A41B 1/08

(57) ABSTRACT

A clothing article including a tourniquet disclosed. In one implementation, the clothing article is worn high at the waist or on the leg. The clothing article includes a belt. The belt presents a holster having a tourniquet. The tourniquet provides a tourniquet strap, a windlass rod and a V-ring. The tourniquet strap mounts to the holster and the windlass rod mounts to the tourniquet strap. When a user gets injured on the leg, the user tightens the tourniquet strap by twisting the windlass rod to further tighten the tourniquet strap such that bleeding stops from the injured area. Further, the V-ring locks the position of the windlass rod once it is determined that adequate pressure is achieved on the leg. In another implementation, the clothing article comes in a vest configuration and the tourniquet mounts at sleeves of the clothing article. The tourniquet helps to stop blood flow to the injured area on the arm.

The present application claims the benefit of U.S. provisional Application No. 62/979,374, filed Feb. 20, 2020, all of which is incorporated herein by reference.

### FIELD OF INVENTION

The present disclosure generally relates to medical devices such as tourniquets. More specifically, the present disclosure relates to a tourniquet built into a clothing article either at an arm or leg portion of the clothing article for stopping blood flow to a wound.

### BACKGROUND OF INVENTION

It is known that tourniquets are used for stopping blood flow to wounds. A tourniquet is a tightly tied band applied around a body part such as an arm or leg in an attempt to stop bleeding or uncontrolled hemorrhage in an emergency situation. Typically, the tourniquet includes a piece of strap or tube wrapped around the arm or leg and tied tightly. Further, the tourniquet includes a windlass or stick wound underneath the strap. In the emergency situation, the strap is wrapped around the limb and the windlass is twisted until the strap is tightened enough such that the bleeding is stopped. Once it is determined that adequate pressure is achieved, the windlass is secured to lock its position.

Generally, the tourniquets are stored in a container, also referred to as a tourniquet storage device. The tourniquet storage devices are made up of a rigid or a non-rigid structure. Further, each of the tourniquet storage devices includes multiple compartments in various configurations for storing the tourniquets. During an emergency situation, an injured person struggles to retrieve a tourniquet from the tourniquet storage device. Delay in retrieving and application of the tourniquet to stop the bleeding results in a serious injury or causes death. The above situation becomes worse if a person carrying the tourniquet storage device is far away from the injured person.

In order to overcome the delay caused due to the retrieval and application of the tourniquet, several solutions have been provided in the past. One such solution includes providing a tourniquet affixed to a clothing article. An example of such a solution is disclosed in a United States granted U.S. Pat. No. 7,604,651, entitled “Military clothing having built-in tourniquets” (the “'651 Patent”). The '651 Patent discloses an article of clothing, body armor or garment having at least one limb-receiving sleeve includes a housing secured in encircling relation to the sleeve. A self-sustaining tourniquet is disposed within the housing. An opening is formed in the housing and a cover is provided to close the opening. The tourniquet is positioned fully within the housing when the cover is closed. The tourniquet is oriented in the housing so that a free end of the tourniquet is in registration with the opening. A handle is secured to the free end and is adapted to be grasped by a user when the cover is open. The diameter of the tourniquet is reduced when the handle is pulled upon. A ratchet and pawl structure prevents the tourniquet from loosening after the handle has been pulled.

Another example is disclosed in a United States granted patent No. 8465514, entitled “Tourniquet system” (the “'514 Patent”). The '514 Patent discloses a tourniquet system having at least one circumferential enclosed channel formed in a garment; a tourniquet band positioned in the at least one circumferential enclosed channel; a tightening and securing mechanism associated with the tourniquet band, wherein the tightening and securing mechanism includes two or more apertures formed therethrough, and wherein at least a portion of the tourniquet band loops through the apertures of the tightening and securing mechanism, such that when the tightening and securing mechanism is turned, a diameter of the tourniquet band is decreased and a tourniquet force is applied to an inside wall of the circumferential enclosed channel; and at least one portal in the garment, proximate the circumferential enclosed channel, to provide access to the tightening and securing mechanism.

Yet another example is disclosed in a United States granted patent No. 7981135, entitled “Garment with affixed tourniquet” (the “'135 Patent”). The '135 Patent discloses an article of clothing having a tourniquet affixed to the clothing at one end of the tourniquet, the tourniquet comprising a band comprised of an elastic and flexible material, the band having a width and a length, a first end and a second end, and a first face and second face, where at least a portion of the first face comprises hook-type fastener means, and where the second face comprises loop-type fastener means.

Although the disclosures disclose a tourniquet affixed to a clothing article that allows an injured person to quickly deploy or put on the tourniquet to stop the bleeding, they have few problems. For instance, the above-discussed disclosures allow storing only one tourniquet. If the person gets injured in more than one leg or arm, then the injured person may have to depend on emergency personnel to arrive or wait for the person carrying the tourniquet storage device to provide him with a tourniquet thereby delaying stopping of the bleeding from the wound.

Therefore, there is a need for providing a tourniquet built into a clothing article, which simplifies application or putting of the tourniquet and shortens the time between the infliction of a wound and application of the tourniquet.

### SUMMARY

It is an object of the present disclosure to provide a tourniquet built into a clothing article and that avoids the drawback of known drain tourniquets.

In order to achieve the object, the present disclosure provides a clothing article having a tourniquet, which simplifies application or putting of the tourniquet and shortens the time between the infliction of a wound or injury and application of the tourniquet.

In one aspect of the present disclosure, a user wears the clothing article on high at the waist or on the leg. In one example, the user includes an emergency responder or military person. The clothing article includes a belt. The belt presents a holster having a holster or holster pouch or magazine pouch. At the end of the holster, the clothing article has a tourniquet. The tourniquet includes a tourniquet strap, a windlass rod and a V-ring. The tourniquet strap encompasses a buckle or hook and loop such as a Velcro for tightening the tourniquet strap on the arm or leg. The tourniquet strap mounts at the holster. Further, the windlass rod mounts to the tourniquet strap. When the user gets injured on the leg, the user twists the windlass rod to further tighten the tourniquet strap such that bleeding stops from the injured area. Further, the V-ring helps to lock the position of the windlass rod once it is determined that adequate pressure is achieved on the leg.

In another aspect of the present disclosure, a clothing article encompassing a tourniquet is disclosed. In the present aspect, the clothing article comes in a vest configuration in that the clothing article is worn either directly on the body or over another clothing article such as a shirt or military uniform and so on worn by the user. In the present aspect, the tourniquet mounts at each sleeve of the clothing article. The tourniquet includes a tourniquet strap, a windlass rod and a V-ring. The tourniquet strap encompasses a buckle or hook and loop such as a Velcro for tightening the tourniquet strap on the arm or leg. The tourniquet strap mounts over the sleeve. Further, the windlass rod mounts to the tourniquet strap. When the user gets injured on arm, the user tightens the tourniquet strap and twists the windlass rod to further tighten the tourniquet strap such that bleeding stops from the injured area on the arm. Further, the V-ring helps to lock the position of the windlass rod once it is determined that adequate pressure is achieved on the arm.

In addition, the clothing article includes straps at the rear side of the clothing article. The straps help to pull the injured user wearing the clothing article from one place to another. In one implementation, the clothing article encompasses a cutaway section that helps to separate the straps, once the user has been pulled to a desired location. Further, the clothing article presents a nameplate to put on the name of the user for easy identification of the user wearing the clothing article.

In one advantageous feature of the present disclosure, the clothing article having the tourniquet shortens the time between the infliction of a wound or injury to bleeding control because the tourniquet is worn as everyday garment. When injury occurs the wearer only has to turn the windlass rod to tighten the tourniquet strap to stop blood flow to the injury. Further, the V-ring can be used to ensure that the windlass rod is properly secured so that the tourniquet strap is tightly put around the arm or leg.

Features and advantages of the subject matter hereof will become more apparent in light of the following detailed description of selected embodiments, as illustrated in the accompanying FIGURES. As will be realized, the subject matter disclosed is capable of modifications in various respects, all without departing from the scope of the subject matter. Accordingly, the drawings and the description are to be regarded as illustrative in nature.

### BRIEF DESCRIPTION OF THE DRAWINGS

With the above and other related objects in view, the invention consists in the details of construction and combination of parts as will be more fully understood from the following description, when read in conjunction with the accompanying drawings in which:

FIG. 1 illustrates a front view of a clothing article including a tourniquet, in accordance with one embodiment of present disclosure;

FIG. 2 illustrates the tourniquet including a windlass rod and a V-ring, in accordance with one embodiment of present disclosure;

FIG. 3 illustrates a front view of a clothing article including a tourniquet, in accordance with another embodiment of present disclosure;

FIG. 4 illustrates a clothing article including a tourniquet, in accordance with another embodiment of the present disclosure;

FIGS. 5 and 7 illustrate a front and a rear view, respectively of a clothing article including a tourniquet, in accordance with another embodiment of the present disclosure;

FIG. 6 illustrates the tourniquet provided on a sleeve of the clothing article; and

FIGS. 8 and 9 illustrate a front and a rear view, respectively of a clothing article including a tourniquet, in accordance with another embodiment of present disclosure.

It will be noted that throughout the appended drawings, like features are identified by like reference numerals.

### DETAILED DESCRIPTION OF THE EMBODIMENTS

Before the present constructional features and working principle of a clothing article including a tourniquet is described, it is to be understood that this disclosure is not limited to the particular clothing article comprising the tourniquet as described, since it may vary within the specification indicated. Various constructional features for the clothing article comprising the tourniquet might be provided by introducing variations within the components/subcomponents disclosed herein. It is also to be understood that the terminology used in the description is for the purpose of describing the particular versions or embodiments only, and is not intended to limit the scope of the present invention or disclosure, which will be limited only by the appended claims. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

It should be understood that the present disclosure describes a clothing article including a tourniquet. In one implementation, the clothing article is worn high at the waist or on the leg. The clothing article includes a belt. The belt presents a holster having a holster pouch or magazine pouches. At one end of the holster, the clothing article provides a tourniquet. The tourniquet includes a tourniquet strap, a windlass rod and a V-ring. The tourniquet strap mounts at the holster. Further, the windlass rod connects to the tourniquet strap. When a user gets injured on the leg, the user tightens the tourniquet strap by twisting the windlass rod to further tighten the tourniquet strap such that bleeding stops from the injured area. Further, the V-ring allows to lock the position of the windlass rod.

In another implementation, the clothing article comes in in a vest configuration. Here, the tourniquet mounts at sleeves of the clothing article. The tourniquet helps to stop blood flow to the injured area on the arm.

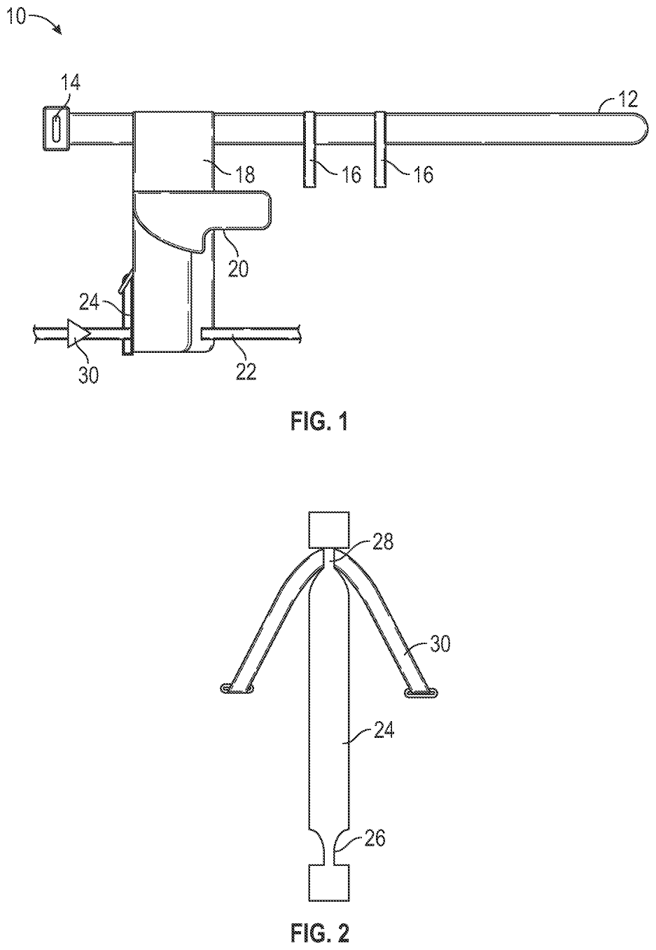

Various features and embodiments of a clothing article including a tourniquet are explained in conjunction with the description of FIGS. 1-9.

FIG. 1 shows a front view of clothing article 10 having a tourniquet, in accordance with one embodiment of the present disclosure. Clothing article 10 includes belt 12. In one example, belt 12 comes in the configuration of a waist belt. As such, belt 12 encompasses groove 14 to draw belt 12 through. Further, belt 12 comprises straps 16. Straps 16 provides a material made of an elastic material or nylon or any other suitable material. A person skilled in the art understands that straps 16 come in either short or long configuration depending on the need.

Clothing article 10 presents a holster 18 extending from the belt 12. Holster 18 provides a material made of a fabric, leather or any other suitable material. Holster 18 encompasses a holster pouch 20. Holster pouch 20 comes in the shape of a firearm or gun such that holster pouch 20 holds or stores the firearm. Further, clothing article 10 includes a tourniquet. The tourniquet comprises tourniquet strap 22. As can be seen in FIG. 1, tourniquet strap 22 mounts at one end of the holster 18. In one example, tourniquet strap 22 encompasses a buckle (not shown) for tightening tourniquet strap 22 around a leg of a user when the user wears clothing article 10. Alternatively, tourniquet strap 22 includes a hook and loop such as a Velcro™ for tightening tourniquet strap 22 around the leg of the user. Further, the tourniquet includes a windlass or windlass rod or stick 24. In one implementation, windlass rod 24 mounts in a vertical configuration with respect to holster 18 (FIG. 1). In one example, windlass rod 24 mounts to tourniquet strap 22 (FIG. 1).

FIG. 2 shows a front view of windlass rod 24, in accordance with one embodiment of the present disclosure. Windlass rod 24 indicates an elongated rod made of metal, hard plastic or any other suitable material. In the current embodiment, windlass rod 24 encompasses grooves or cut portions i.e., first groove 26 and second groove 28 at opposite ends. In other words, windlass rod 24 encompasses first groove 26 at one end and second groove 28 at other end. Further, the tourniquet includes V-ring or V-shaped ring 30. Specifically, windlass rod 24 presents V-ring 30. V-ring 30 provides a material made of metal, plastic or any other suitable material. In operation, V-ring 30 engages in the grooves i.e., either at first groove 26 or second groove 28 and locks the position of windlass rod 24.

Now referring to FIGS. 1 and 2, the use of clothing article 10 including the tourniquet is explained. In order to use clothing article 10, the user wears belt 12 at waist high thereby suspending holster 18 and positioning holster 18 over leg i.e., thigh portion of the user. When the user gets injured on the leg, the user uses the tourniquet i.e., tourniquet strap 22, windlass rod 24 and V-ring 30 to stop bleeding from the injury. In order to stop bleeding from the injury, at first, the user tightens tourniquet strap 22 around the leg. Subsequently, the user twists or rotates windlass rod 24 until tourniquet strap 22 is tightened enough ensuring that the bleeding is stopped from the injury. After tightening tourniquet strap 22, position of the windlass rod 24 is locked by placing V-ring 30 in one of first groove 26 and second groove 28. In other words, the user twists windlass rod 24 and places V-ring 30 in one of first groove 26 and second groove 28 to lock the position of windlass rod 24 such that tourniquet strap 22 is held tightly around the leg of the user once it is determined that adequate pressure is achieved on the leg.

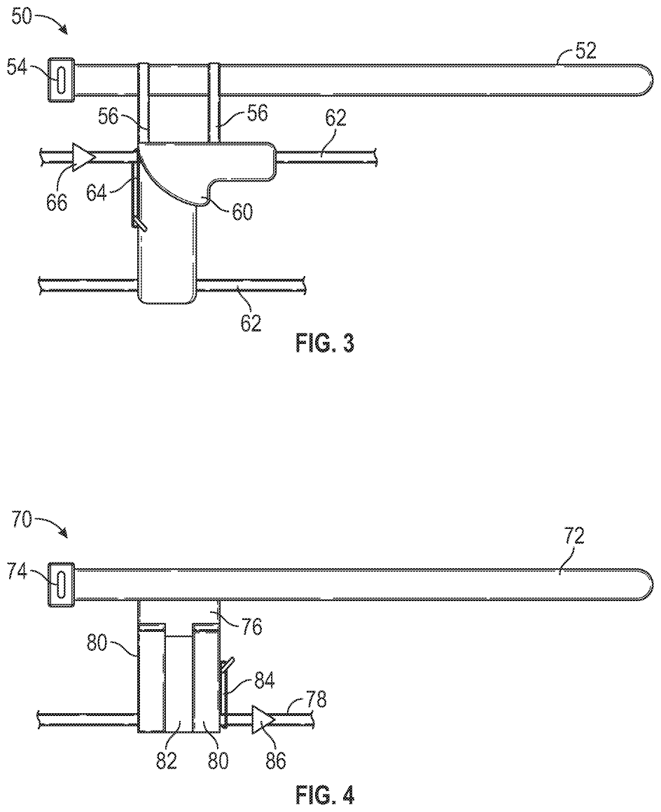

FIG. 3 shows a front view of clothing article 50 including a tourniquet, in accordance with another embodiment of the present disclosure. Clothing article 50 includes belt 52 (similar to belt 12 shown in FIG. 1). Belt 52 encompasses groove 54 to draw belt 52 through. Further, belt 52 presents straps 56. In the current embodiment, clothing article 50 encompasses holster pouch 60 that connects to straps 56. Holster pouch 60 comes in the shape of a firearm or gun such that holster pouch 20 holds or stores the firearm. Further, clothing article 50 includes a tourniquet. The tourniquet includes tourniquet strap 62. As can be seen in FIG. 3, tourniquet strap 62 mounts at one end of holster pouch 60 or straps 56. In one example, tourniquet strap 62 includes a buckle (not shown) for tightening tourniquet strap 62 around a leg of a user when the user wears clothing article 50. Alternatively, tourniquet strap 62 includes a hook and loop such as a Velcro™ for tightening tourniquet strap 62 around the leg of the user. Further, the tourniquet includes a windlass rod 64 (similar to windlass rod 24 shown in FIGS. 1 and 2). Further, tourniquet comprises V-ring 66 to lock the position of windlass rod 64, as explained above.

It should be understood that the construction of clothing article 50 shown in FIG. 3 is different from clothing article 10 shown in FIG. 1, in that holster 18 extends from belt 12 and holds the tourniquet in FIG. 1, and in FIG. 3, holster pouch 60 holding the tourniquet mounts to straps 56 extending from belt 52. Further, a person skilled in the art understands that the function of clothing article 50 is same as clothing article 10. As explained above, a user wears belt 52 on his leg suspending holster pouch 60. When the user gets injured, the user uses the tourniquet i.e., tourniquet strap 62, windlass rod 64 and V-ring 66 to stop bleeding from the injury. In order to stop bleeding from the injury, the user tightens tourniquet strap 62 and twists or rotates windlass rod 64 until tourniquet strap 62 is tightened enough to ensure that the bleeding is stopped from the injury. As explained above, windlass rod 64 twists and V-ring 66 locks the position of windlass rod 64 to hold tourniquet strap 62 firmly in position.

FIG. 4 shows clothing article 70 including a tourniquet, in accordance with another embodiment of the present disclosure. Clothing article 70 includes a belt 72 (similar to belt 12 shown in FIG. 1). Belt 72 includes groove 74 to draw belt 72 through. Further, belt 72 presents straps 76. In the current embodiment, clothing article 70 encompasses holster 76 extending from belt 72. In the current embodiment, clothing article 70 presents magazine pouches 80. Magazine pouches 80 indicate pouches or pockets for storing gun magazines or flashlight, radio, etc. Magazine pouches 80 provide material made of fabric or leather or plastic or any other suitable material. Magazine pouches 80 connect via connecting means 82. Connecting means 82 indicate a fabric or any other material provided between magazine pouches 80 to connect them.

Further, clothing article 70 includes a tourniquet. The tourniquet includes tourniquet strap 78. As can be seen in FIG. 4, tourniquet strap 78 mounts at one end of holster 76. In one example, tourniquet strap 78 includes a buckle (not shown) or a hook and loop such as a Velcro™ for tightening tourniquet strap 78 around a leg of a user. Further, the tourniquet includes windlass rod 84 (similar to windlass rod 24 shown in FIGS. 1 and 2). The tourniquet includes V-ring 86 to lock the position of windlass rod 84.

In order to use clothing article 70 for stopping bleeding from an injury, a user wears belt 72 on his leg suspending holster 76 from belt 72. When the user gets injured, the user uses the tourniquet i.e., tourniquet strap 78, windlass rod 84 and V-ring 86 to stop bleeding from the injury. In order to stop bleeding from the injury, the user tightens the tourniquet strap 78 and twists the windlass rod 84 until the tourniquet strap 78 tightens enough to ensure that the bleeding is stopped from the injury. As explained above, windlass rod 84 twists and V-ring 86 locks the position of the windlass rod 84 such that tourniquet strap 78 is held tightly around the leg of the user once it is determined that adequate pressure is achieved on the leg.

In one implementation, the user uses clothing article 70 either separately, or along with clothing article 10 or clothing article 50. In order to use clothing article 70 along with clothing article 10, the user puts on holster 76 on one leg and holster 18 on another. Whenever there is an injury, the user uses the tourniquet provided at respective holster 76 or 18. For example, the user puts on clothing article 10 on the right leg and clothing article 70 on the left leg. When there is an injury on the right leg, the user tightens tourniquet strap 22 and uses windlass rod 24 and V-ring 30 to further tighten tourniquet strap 22 to stop bleeding from the injury, as explained above. Similarly, when there is an injury on the left leg, the user tightens tourniquet strap 78 and uses windlass rod 84 and V-ring 86 to further tighten tourniquet strap 78 to stop bleeding from the injury, as explained above.

A person skilled in the art appreciates that clothing article 10 mounts at a higher portion of the legs and clothing article 50 mounts at a lower portion of the legs, however the use of the clothing articles should not be construed in a limited sense. Further, a person skilled in the art appreciates that each of clothing articles 10, 50 and 70 includes a tourniquet that helps to stop bleeding from an injury on legs. Although it is shown that the clothing articles come in the form of holsters, a person skilled in the art appreciates that the clothing article may come in the form of shorts or trousers with the tourniquet explained above. Such clothing articles having the tourniquet come in one or both leg portions of the clothing article to stop bleeding from injury on one leg or injuries on both legs.

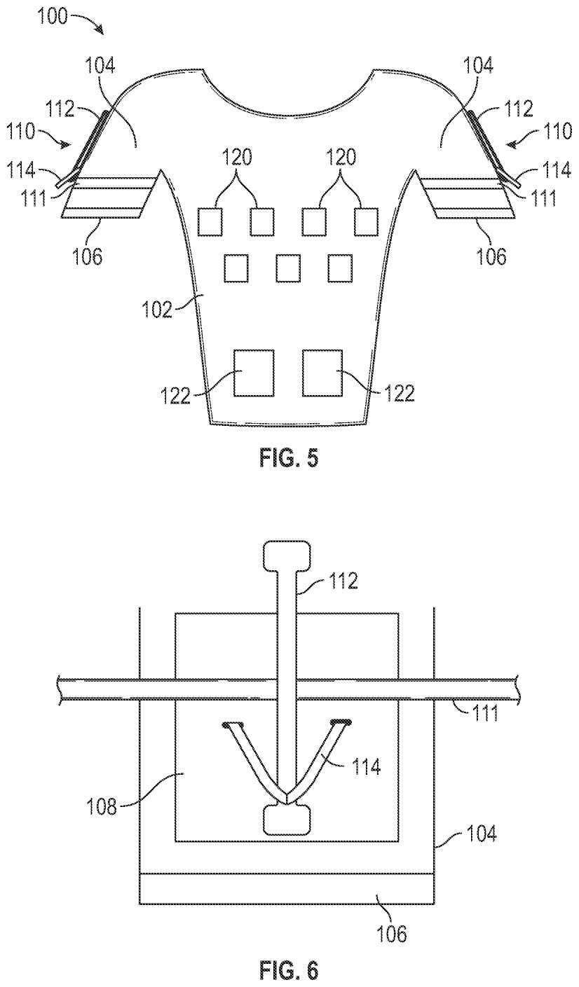

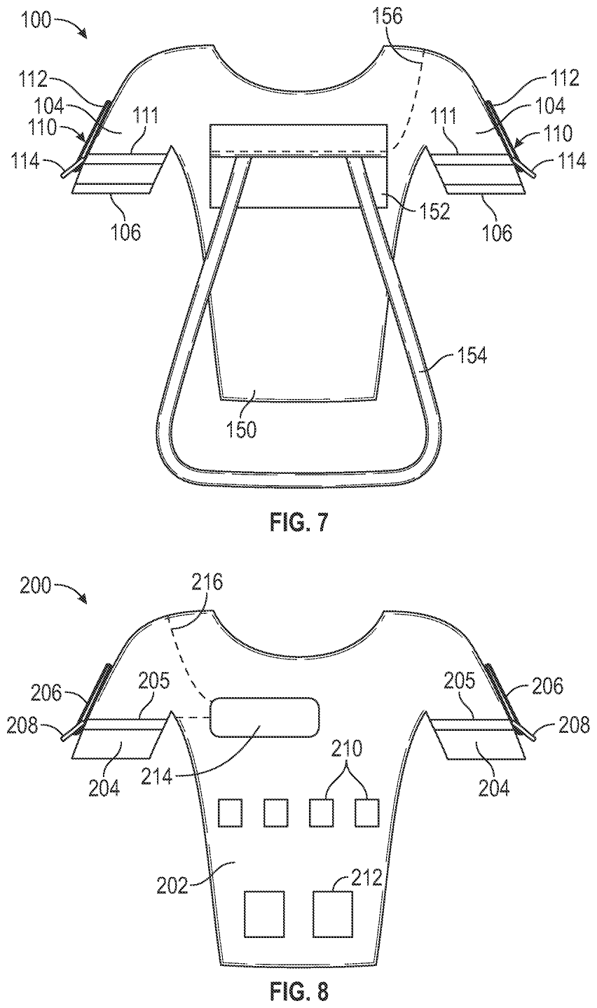

FIGS. 5 and 7 show a front and a rear view, respectively of clothing article 100 including tourniquet 110, in accordance with one embodiment of the present disclosure. In the current embodiment, clothing article 100 comes in a vest configuration that can be worn by a user either directly on the body or over another clothing article such as a shirt or military uniform and so on. Clothing article 100 includes a front portion or a vest portion 102 and sleeves 104. In one example, each of the sleeves 104 includes elastic cuff 106 at the end of sleeve 104. Further, each of sleeves 104 presents anti-pinch plate 108. Anti-pinch plate 108 provides a material made of plastic, metal or any other suitable material. Anti-pinch plate 108 helps the user from getting hurt when tourniquet 110 is tightened as the anti-pinch plate 108 comes in between the skin/clothing of the user and the tourniquet 110.

In accordance with the current embodiment, each of the sleeves 104 presents tourniquet 110. FIG. 6 shows sleeve 104 having tourniquet 110. In one example, tourniquet 110 mounts in a vertical configuration with respect to sleeve 106. Tourniquet 110 includes tourniquet strap 111. Further, tourniquet 110 includes a windlass rod 112 and V-ring 114. As can be seen, windlass rod 112 mounts to tourniquet strap 111. Further, clothing article 100 encompasses pockets 120 and pouches 122 at front portion 102. Pouch 122 indicates a quick clot pouch used for holding chemicals or any other objects.

FIG. 7 shows rear portion 150 of clothing article 100. At rear portion 150, clothing article 100 provides a nameplate portion 152 for putting on the name of the user such that emergency personnel or others will know the identity of the user in case of an emergency situation or when the user gets injured. Nameplate portion 152 provides a material made of a fabric or rubber or any other suitable material. Further, clothing article 100 includes straps 154 extending from rear portion 150. Straps 154 help in pulling an injured user wearing the clothing article 100 from one place to another. In one example, clothing article 100 comes with a cutaway cord 156 that helps to separate straps 154, once the user has been pulled to a desired location.

In the current embodiment, when an arm of the user gets injured, the user tightens windlass rod 112. As specified above, tourniquet strap 111 includes a buckle or hook and loop such as Velcro, which helps to tighten the tourniquet strap 111 around the arm. Subsequently, the user twists windlass rod 112 until tourniquet strap 111 tightens enough to stop the blood from the injury. Further, V-ring 114 helps to lock the position of the windlass rod 112 once it is determined that adequate pressure is achieved on the arm by tourniquet strap 111. Concurrently or consecutively, emergency personnel or other bystanders use straps 154 to pull the user e.g., proximity to an ambulance or safe area. Further, the emergency personnel use cutaway cord 156 to separate straps 154 from clothing article 100 and provide further medical care needed to the injured user of clothing article 100.

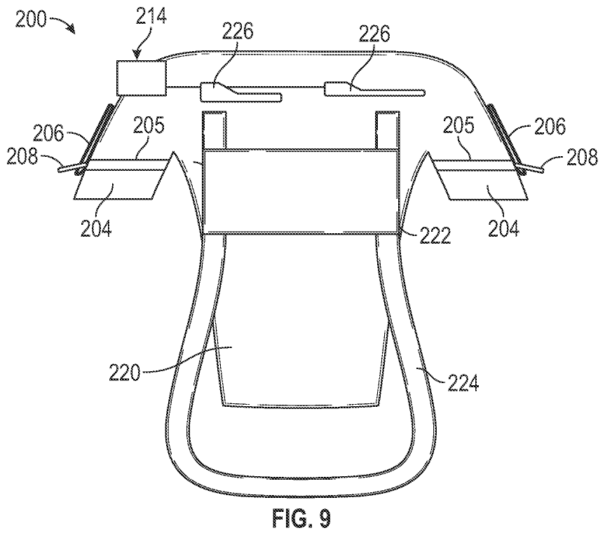

FIGS. 8 and 9 show a front and a rear view, respectively of clothing article 200 including a tourniquet, in accordance with another embodiment of the present disclosure. In the current embodiment, clothing article 200 comes in a vest configuration that can be worn by the user either directly on the body or over another clothing article such as a shirt or military uniform and so on. Clothing article 200 includes front portion or vest portion 202 and sleeves 204. In the current embodiment, each of the sleeves 204 includes a tourniquet. The tourniquet comprises tourniquet strap 205, windlass rod 206 and V-ring 208. Further, clothing article 200 provides pockets 210 and pouches 212 at front portion 202. Further, clothing article 200 encompasses front nameplate portion 214 for putting on the name of the user. In one example, clothing article 100 presents cutaway cord 216 that helps to separate straps 224 provided at rear portion 220 of clothing article 200.

Further, clothing article 200 includes rear portion 220. At rear portion 220, clothing article 100 provides rear nameplate portion 222 for putting on the name of the user. Further, clothing article 200 includes straps 224 extending from rear portion 220. Straps 224 help in pulling an injured user wearing clothing article 100 from one place to another. Further, clothing article 200 includes pins 226 for pulling front nameplate portion 214. A person skilled in the art understands that releasing pins 226 allows the straps 224 to be removed.

In the current embodiment, when an arm of the user gets injured, the user tightens the tourniquet by twisting windlass rod 206 until tourniquet strap 205 tightens enough to stop the blood from the injury. Further, V-ring 208 locks the position of windlass rod 206 such that tourniquet strap 205 is held tightly around the leg of the user once it is determined that adequate pressure is achieved on the leg. Concurrently or consecutively, emergency personnel or others use straps 224 to pull the user e.g., proximity to an ambulance or safe area. Further, the emergency personnel pull front nameplate portion 214, release pins 226 to separate straps 224 from clothing article 200 and provide further medical care needed to the injured user of clothing article 200.

A person skilled in the art understands that the user wears clothing articles 100 or 200 over shirts or uniforms and uses the tourniquet to stop bleeding from the arm. Further, as the tourniquet is provided at the sleeves of the clothing article, it is easier to tighten the tourniquet to stop the bleeding. Further, the V-ring ensures that the windlass rod is properly secured so that the tourniquet strap is tightly put around the arm thereby stopping the blood flow to the injury on the arm. Further, clothing articles 100 or 200 help to drag the user with the help of straps 154 and 224 provided at the rear of clothing articles 100 and 200, respectively.

Based on the above, it is evident the clothing article including the tourniquet is easy to use in the event of the user is injured. The user puts on the tourniquet strap and twists the windlass rod to tighten the tourniquet strap around the leg or arm. Further, the V-ring ensures that the windlass rod is properly secured so that the tourniquet strap is tightly put around the arm/leg to stop the blood flow to the injury. As the user will be able to quickly install the tourniquet around the arm/leg, the presently disclosed tourniquet helps to quickly apply pressure around the arm or leg portion of the user in order to stop bleeding from the injury. As such, the tourniquet shortens the time between the infliction of a wound and bleeding control.

Although the above disclosure is explained considering that the tourniquet is built into a clothing article, a person skilled in the art will appreciate that the tourniquet might be provided as a separate component. The tourniquet comprising the tourniquet strap, the windlass rod and V-ring might be provided as separate apparatus and the tourniquet might be used to stop blood flow to the injury on arm or leg using the explanation provided above.

It should be understood that the shape, size and placement of each component shown in figures are provided for illustrative purpose only and should not be construed in a limited sense. A person skilled in the art will appreciate alternate parts and/or mechanisms that might be used to implement the embodiments of the present invention/disclosure and such implementations will be within the scope of the present disclosure.

While preferred embodiments have been described above and illustrated in the accompanying drawings, it will be evident to those skilled in the art that modifications may be made without departing from this disclosure. Such modifications are considered as possible variants within the scope of the disclosure.

What is claimed is:

1. A tourniquet comprising:
a tourniquet strap;
a windlass rod, wherein said windlass rod comprises a first groove and a second groove at opposite ends; and
a V-ring connecting said windlass rod, wherein
said V-ring operates between said first groove and said second groove,
wherein said tourniquet strap mounts around an arm or leg portion of a user upon getting injured,
wherein said windlass rod twists to tighten said tourniquet strap around the arm or leg portion of the user, and wherein said V-ring positions in one of the first groove and the second groove to tightly hold said windlass rod such that said tourniquet strap holds tightly and applies pressure around the arm or leg portion of the user in order to stop bleeding from the injury.

2. The tourniquet of claim 1, wherein the tourniquet strap mounts to a holster, wherein the holster connects to a belt, and wherein the belt connects at a waist high of the user and allows for suspending the holster and positioning the tourniquet strap at the leg portion of the user.

3. The tourniquet of claim 1, wherein the tourniquet strap mounts to a holster, wherein the holster connects to a belt via straps, and wherein the belt connects at a waist high of the user and allows for suspending the holster and positioning the tourniquet strap at the leg portion of the user.

4. The tourniquet of claim 1, wherein the tourniquet strap mounts to magazine pouches, wherein the magazine pouches connect to a belt via straps, and wherein the belt connects at a waist high of the user and allows for suspending the magazine pouches and positioning the tourniquet strap at the leg portion of the user.

5. The tourniquet of claim 1, wherein the tourniquet further comprises a belt connecting at a waist high of the user, wherein the belt comprises a holster and magazine pouches that suspend from the belt and position the tourniquet strap at the leg portion of the user, and wherein the tourniquet strap connects to the holster and the magazine pouches.

6. The tourniquet of claim 1, wherein the tourniquet strap mounts around a sleeve of a clothing article worn by the user.

7. The tourniquet of claim 1, wherein the tourniquet strap mounts around a sleeve of a clothing article worn by the user, and wherein the clothing article is a shirt or vest.

8. The tourniquet of claim 1, wherein the tourniquet strap mounts around a sleeve of a clothing article, and wherein the clothing article comprises straps for pulling the user wearing the clothing article.

9. The tourniquet of claim 1, wherein the tourniquet strap mounts around a sleeve of a clothing article, wherein the clothing article comprises straps, and a cutaway cord connecting the straps, and wherein the straps allow to pull the user wearing the clothing article from one place to another and the cutaway cord helps to separate the straps from the clothing article upon pulling the user.

10. The tourniquet of claim 1, wherein the tourniquet strap comprises a buckle or hook and loops, and wherein the buckle or the hook and loops help to tighten the tourniquet strap around the arm or leg portion of the user.

11. A method of providing a tourniquet, the method comprising steps of:
providing a tourniquet strap;
providing a windlass rod having a first groove and a second groove at opposite ends;
providing a V-ring at said windlass rod;
mounting said tourniquet strap around an arm or leg portion of a user upon getting injured;
twisting said windlass rod for tightening said tourniquet strap around the arm or leg portion of the user; and
locking the position of said windlass rod by positioning said V-ring in one of the first groove and the second groove, and wherein locking the position of said windlass rods holds said tourniquet strap tightly, applies pressure around the arm or leg portion of the user and stops bleeding from the injury.

12. The method of claim 11, further comprising:
connecting the tourniquet strap to a holster, the holster connecting a belt worn at a waist high of the user; and
suspending the holster from the belt and positioning the tourniquet strap at the leg portion of the user.

13. The method of claim 11, further comprising:
connecting the tourniquet strap to a holster, the holster connecting a belt via straps, the belt worn at a waist high of the user; and
suspending the holster from the belt and positioning the tourniquet strap at the leg portion of the user.

14. The method of claim 11, further comprising:
connecting the tourniquet strap to magazine pouches, the magazine pouches connecting to a belt worn at a waist high of the user; and
suspending the magazine pouches from the belt and positioning the tourniquet strap at the leg portion of the user.

15. The method of claim 11, further comprising:
connecting the tourniquet strap to a belt worn at a waist high of the user;
providing a holster and magazine pouches at the belt; and
suspending the holster and the magazine pouches from the belt and positioning the tourniquet strap at the leg portion of the user.

16. The method of claim 11, further comprising mounting the tourniquet strap around a sleeve of a clothing article worn by the user.

17. The method of claim 11, further comprising mounting the tourniquet strap around a sleeve of a clothing article, the clothing article having straps for pulling the user wearing the clothing article.

18. The method of claim 11, further comprising:
mounting the tourniquet strap around a sleeve of a clothing article;
providing straps and a cutaway cord connecting the straps at the clothing article;
pulling the user wearing the clothing article using straps; and
separating the straps upon pulling the user using the cutaway cord.

19. The method of claim 11, further comprising providing a buckle or hook and loops at the tourniquet strap for tightening the tourniquet strap around the arm or leg portion of the user.
